(19) United States
(12) Patent Application Publication
(10) Pub. No.: US 2023/0302218 A1
(43) Pub. Date: Sep. 28, 2023

(54) CONTROL DEVICE FOR A PERISTALTIC PUMP, PERISTALTIC PUMP, INJECTION APPARATUS AND METHOD FOR CONTROLLING A PERISTALTIC PUMP

(71) Applicant: Ulrich GmbH & Co. KG, Ulm (DE)

(72) Inventors: ANDREAS BECKER, Ulm (DE); Felix Seibold, Ulm (DE)

(21) Appl. No.: 18/126,643

(22) Filed: Mar. 27, 2023

(30) Foreign Application Priority Data
Mar. 25, 2022 (DE) 102022107119.7

(51) Int. Cl. A61M 5/142; F04B 43/12; F04B 49/20

(52) U.S. Cl. CPC A61M 5/14232; F04B 43/1253; F04B 49/20; A61M 5/007

(57) ABSTRACT

Disclosed is a control device for a peristaltic pump which delivers a medium in pulsating pressure cycles, the control device controlling a speed of the peristaltic pump in such a way that a maximum volume flow (Qₘₐₓ) is achieved without exceeding a pressure limit (p_{Grenz}) in the delivery line. In certain embodiments, it is proposed to calculate a prediction pressure (p_{futur}) for each pressure cycle (P) for an expected maximum pressure within the pressure cycle (P) on the basis of at least the pressure (pᵢₛₜ) in the output line and to limit the maximum volume flow (Qₘₐₓ) by using the prediction pressure so that the pressure (pᵢₛₜ) in the output line does not exceed the pressure limit (p_{Grenz}) in the pressure cycle (P).

The invention relates to a control device for a peristaltic pump according to the preamble of claim 1, a peristaltic pump with such a control device, an injection device with such a peristaltic pump and a method for controlling a peristaltic pump.

Injection devices are medical devices by means of which liquid injection agents (medium) can be introduced into a human or animal body in a controlled manner via a pumping device. The injection medium can be, for example, a contrast medium for increasing the contrast in an imaging procedure such as computer tomography or magnetic resonance imaging. In addition to controlling the injection volume as well as a flow rate (volumetric flow) of the injection agent, it is necessary to monitor the pressure in a discharge line, since excessively high pressures can on the one hand be harmful to the body and/or damage the injection device. For this reason, known injection devices provide controls or regulators that switch off the injection device or its pumping device when a still permissible pressure limit (maximum set pressure), which is below a defined hazard pressure, is exceeded. Especially in the case of cyclically oscillating pressure curves, which are present e.g. in axial piston or roller pumps, pressure maxima can occur which are only briefly and slightly above the pressure limit and therefore lead to an unnecessary shutdown of the pumping device and a termination of the injection process. This prolongs the injection process and unnecessarily increases the total injected volume of injection agent.

In order to avoid premature termination of an injection process, it is proposed in U.S. Pat. No. 6,673,033 B1 to define an intermediate pressure threshold, above which the output of a pumping device is initially throttled, and to switch off the pumping device only if the pressure in a hose line of an injector subsequently rises above a pressure limit value despite the throttled output.

Similarly, it is known from DE 10 2013 113 387 A1 that instead of a hard pressure limit value as a criterion for aborting an injection process in a peristaltic pump, a temporal integral of the pressure curve from exceeding a pressure limit value is used as a criterion for aborting the injection process, so that short-term pressure peaks can be tolerated and only briefly exceeding the pressure limit value does not lead to an immediate abort of the injection process.

A disadvantage of the control methods for injection devices known from the state of the art is that large safety intervals must be observed—i.e. the pressure limit must be significantly below the hazard pressure—so that there is sufficient reaction time to be able to regulate the pump device down or switch it off when the pressure limit is exceeded.

Against this background, it is the objective of the invention to specify an improved control device for a peristaltic pump with an oscillating pressure curve (pressure pulsation), a peristaltic pump with such a control device, an injection device with such a peristaltic pump as well as a control method in which safety intervals can be reduced and a predetermined pressure limit can be brought closer to a hazardous pressure without increasing a risk of damage to the patient and/or the material of the injection device. At the same time, pressure pulsations are to be reduced.

This objective is solved, inter alia, by the control device of claim 1, the peristaltic pump according to claim 14, an injection device according to claim 16, and a control method according to claim 17.

The control device according to the invention is used to control a peristaltic pump with a squeeze tube and cyclically moving conveying elements for conveying a medium guided in the squeeze tube during a conveying action with a controlled volume flow rate into a discharge line connected to the squeeze tube. In doing so, the conveying elements compress the squeeze tube cyclically so that a pressure is established in the output line with a pressure curve that has cyclically repeating pressure cycles. Each pressure cycle can be defined in such a way that it extends, for example, from a pressure minimum via a pressure increase to a pressure maximum and then drops again to a pressure minimum of a subsequent pressure cycle. The control device according to the invention controls a speed of the peristaltic pump—in the case of a roller pump, this can be an angular speed of a conveying element—in such a way that a maximum volumetric flow rate is achieved without exceeding a pressure limit in the discharge line. The pressure limit can be a desired, still permissible set pressure. For this purpose, the control device has at least a first control loop for controlling the maximum volumetric flow rate. This receives a setpoint volumetric flow and the pressure limit as externally specified command variables. The control device is set up to carry out a control method in which a predicted pressure for an expected maximum pressure within the pressure cycle is calculated for each pressure cycle on the basis of at least the pressure in the discharge line and the maximum volumetric flow is limited taking into account the predicted pressure in such a way that the pressure in the discharge line does not exceed the pressure limit in the pressure cycle.

It is expedient for the first control loop to detect the current pressure in the discharge line as a feedback variable, on the basis of which a control section of the control loop regulates the maximum volume flow rate in a predictive manner.

Through predictive control, the speed of the peristaltic pump, in particular the delivery elements of the peristaltic pump, can be proactively adjusted and reduced at an early stage if the pressure limit is about to be exceeded. As reaction times are extended by pressure prediction, safety reserves increase. Pressure limits that are still permissible can then be brought closer to critical pressure values, such as a hazard pressure, which in turn means higher volume flow rates and thus, for example, a shortening of an injection duration for a given injection volume. In addition, as the importance of a fast-reacting mechanism becomes less important with a predictive control, more inert components can be used for the peristaltic pump, which makes the production of the peristaltic pump cheaper. Calibration of the control method is not necessary if the control device is suitably designed.

In a preferred embodiment of the invention, the first control loop comprises a pressure phase detection, by which the end of a preceding pressure cycle and the beginning of a subsequent pressure cycle is detected. The detection can take place on the basis of a characteristic variable of the pressure curve in the output line, in particular a defined pressure drop compared to a pressure maximum of the preceding pressure cycle. The start of a pressure cycle can be defined, for example, at the point in time at which a pressure drop compared to the (absolute) pressure maximum of the previous pressure cycle occurs by more than a certain relative amount, for example, by at least ⅙, ¼ or ⅓ or—independent of the previous pressure maximum—by an absolute amount, e.g. at least 2 bar. The pressure drop is advantageously selected so large that this pressure drop only occurs once towards the end of a pressure cycle. The occurrence of this pressure drop is then sufficiently robust to indicate the start of a subsequent pressure cycle. If a typical pressure cycle has larger pressure fluctuations, larger pressure drops and/or incremental counters that count the number of pressure drops by a certain minimum amount can be used as a substitute in order to recognise the start of a subsequent pressure cycle.

At the beginning of each pressure cycle, the control of the maximum volume flow rate is re-initiated and, in particular, temporarily stored values, such as a temporarily stored pressure maximum of the previous pressure cycle, are updated.

For a further improvement of the control device, it is advantageously provided that each pressure cycle is divided into successive pressure phases on the basis of predefined characteristics and the pressure phase detection recognises the start of the individual pressure phases, whereby the predicted pressure for controlling the maximum volume flow rate in the first control loop is only used in certain pressure phases and is ignored or not used in other pressure phases. The control is thus discontinuous.

An advantage of such a pressure phase-dependent control is that the pressure prediction can be used precisely in the pressure phases in which the development of the pressure in the output line is dynamic and an exceeding of the pressure limit can occur comparatively suddenly, so that in these pressure phases the maximum volume flow rate is controlled conservatively, while in other pressure phases it can be controlled more aggressively, since a sudden exceeding of the pressure limit is not to be expected in these other pressure phases. A further advantage is that different controls can be realised for different pressure phases, e.g. by connecting or disconnecting additional control loops that are superimposed on the first control loop and/or by changing the control parameters of the first control loop, such as amplification factors or similar used therein.

The aforementioned predefined characteristics can be absolute or relative pressure changes and/or an absolute or relative rate of pressure change of the pressure in the output line. The predefined characteristics do not necessarily have to be punctual values. It is possible, for example, to select moving averages over certain periods of time, such as 0.1, 0.2 or 0.5 seconds.

The recognition of the individual pressure phases by the pressure phase recognition can take place on the basis of the predefined characteristics. Alternatively, individual pressure phases can also be recognised by the pressure phase recognition on the basis of a position of the conveying elements of the peristaltic pump, since the positions of the conveying elements correlate with the individual pressure phases, as explained in more detail below. It is also possible to combine the detection based on predefined characteristics and a determination of the position of the conveying elements for the pressure phase detection, either simultaneously in order to make the pressure phase detection of individual pressure phases redundant or in order to predetermine individual pressure phases based on the defined characteristics and other pressure phases based on the position of the conveying elements. Preferably, the pressure phase detection is designed redundantly to increase reliability. This can reduce the susceptibility to errors and, in particular, the requirements for the failure safety of individual components, and thus component costs.

In one embodiment of the invention, the pressure cycle is characterised by five successive pressure phases, wherein a first pressure phase is characterised by a rapid pressure drop, a second pressure phase by a rapid pressure rise, a third pressure phase by a mild pressure rise, a fourth pressure phase by a moderate pressure rise and a fifth pressure phase by a pressure plateau with substantially constant pressure, and each pressure phase is detected by the pressure phase detection. Such a division of a pressure cycle is a good approximation for pressure cycles in injection devices for intravenous injection of e.g. contrast agents. The static and dynamic pressure resistances in the discharge line (and lines connecting thereto) are responsible for the individual pressure phases, which are caused in particular by opening and closing non-return valves, throttles, the mass inertia of the medium to be delivered and other influences. For other applications, the pressure phases can also be defined differently than in the example above.

It is preferable if in the first control loop the predicted pressure is used to control the maximum volume flow rate only in the fourth pressure phase, i.e. the pressure phase before the pressure plateau with the (absolute) pressure maximum within the pressure cycle. The fourth pressure phase is advantageously defined in a range extending from about 50% to about 80% of a phase progress of the pressure cycle. By phase progress is meant the pressure cycle in temporal resolution, i.e. the pressure cycle starts at 0% phase progress—this corresponds e.g. to a pressure minimum—and ends at 100% —this then corresponds to the pressure minimum of the following pressure cycle. Since the predicted pressure is ‘switched on’ to the first control loop in the fourth pressure phase to calculate the maximum volume flow rate, and is otherwise ‘switched off’, it is a discontinuous controller. By limiting the use of the predicted pressure in this way, a complex calculation of the predicted pressure in the other pressure phases can be dispensed with.

In a simple and therefore preferable variant, the prediction pressure can be determined via a linear extrapolation of the current pressure increase in the output line to a specific (calculated) phase progress, which preferably lies in the range of 80% to 95% of the total phase progress and can lie, for example, at 80%, 90% or 95% of the total phase progress and in particular at 87% of the total phase progress. When using the prediction pressure only in the fourth pressure phase, the prediction accuracy of a linear extrapolation is sufficiently accurate even with a strongly oscillating pressure course of a pressure cycle. Other calculation methods for determining the prediction pressure, especially extrapolation based on a time series, are also possible.

The determined (calculated) phase progress, which is used to determine the prediction pressure, is appropriately fixed and is, for example, a constant 87% of the total phase progress. This determined phase progress can—but does not have to—correspond to the real phase progress, where real pressure maxima occur in the pressure cycles. Actual pressure maxima can therefore occur at actual phase advances that lie before or also behind the specific (calculated) phase progress of 87% of the total phase progress in the example.

The determined phase progress is preferably selected and adjusted by calculation in such a way that predicted pressures are achieved which reflect the actual pressure maxima of successive pressure cycles as accurately as possible. The determined (calculated) phase progress to which the prediction pressure is extrapolated can be shifted to the real phase progress at which a real pressure maximum occurs, if this results in better prediction pressures.

In a more specific application of the invention, the control device controls a roller pump. Roller pumps have the advantage of non-occurring changes in direction, which is why roller pumps are particularly well suited for precise dosing of injection boluses in injection devices. Since the pumped medium is conveyed in a squeeze tube in roller pumps, there is no direct contact between the pumped medium and the pump, which is advantageous for hygienic reasons. Furthermore, the squeeze tube can be easily replaced.

It is therefore also an object of the invention to provide a roller pump with a rotatable rotor, a plurality of conveying elements for squeezing a squeeze tube, which are in the form of rollers (squeeze rollers) arranged on the rotor, wherein the squeeze tube is supported along a tube bed with an inlet region and an outlet region and the conveying elements, upon rotation of the rotor, each compress in a fluid-tight manner a section of the squeeze tube when the conveying elements enter the tube bed at the level of the inlet region and until the conveying elements exit the tube bed at the level of the outlet region and thereby a medium located in the squeeze tube is conveyed in the direction of rotation against a dynamic pressure arising in a discharge line connected to the squeeze tube into the discharge line, wherein the roller pump contains a control device according to the invention.

In each case, a volume of the squeeze tube located upstream of the outlet area is compressed from a maximum volume to a minimum volume until a conveying element emerges from the tube bed at the level of the outlet area, releases the section of the squeeze tube compressed by this conveying element and thus increases the volume of the squeeze tube located upstream of the outlet area from the minimum volume to the maximum volume. The volume reduction and volume increase of the volume located upstream of the outlet area constitute a complete pressure cycle.

The roller pump further has n preferably radially equally distributed delivery elements, so that the roller pump performs n pump strokes with n pressure cycles during a complete 360° rotation of the rotor. The number of roller elements is e.g. three.

Since each conveying element is only involved in a pressure cycle in a certain angular section, namely in the angular section in which the conveying element represents the “leading” role in relation to the outlet area or the discharge line, the angular position of the conveying element in this angular section correlates with the phase progress of the pressure cycle. As a result, individual angular ranges of the angular position of the conveying elements within the angular section also correlate with individual pressure phases of a pressure cycle.

This advantageously enables a determination of the pressure phase based on the angular position of one of the conveying elements, especially a ‘leading’ conveying element. Instead of an error-prone pressure phase detection via predefined characteristics, which can be unreliable, it is useful if the pressure phase detection of the control device determines the start of at least one pressure phase of a pressure cycle via an angular position of a conveying element.

In order to be able to do without position sensors for determining the angular position of the conveying elements, the angular position is expediently determined indirectly, e.g. via the time integration of the angular speed of the conveying elements or the rotor of the roller pump. The angular speed of the rotor is usually directly available via a control unit of the roller pump. As shown above, an absolute angular position of the leading conveying element can be approximated via a pressure drop by a minimum amount.

In a further embodiment of the invention, the first control loop comprises a mean value filter that adjusts the maximum volume flow rate depending on a maximum pressure of a pressure cycle immediately preceding the current pressure cycle that is temporarily stored in the control device. This can compensate for errors in the measurement of the pressure in the output line. In addition, the oscillation behaviour of the control loop can be damped by the mean value filter and, in particular, overshooting of the controlled variable can be prevented or at least reduced. For this purpose, the previous, measured maximum pressure is fed back into the first control loop as the ratio of the measured maximum pressure to the pressure limit (set pressure) and a new setpoint volumtric flow is set based on this. A deviation between the actually occurring maximum pressure and the pressure limit (set pressure) can thus be reduced with each cycle.

Advantageously, the first control loop is a PID controller with a proportional element, an integral element and a differential element. A PID controller enables a fast and accurate approximation of the controlled variable to the command variables.

In the differential element, the predicted pressure is preferably processed to control the maximum volume flow rate. However, other feedback variables, e.g. a rate of change of a rotational speed of a rotor of a peristaltic pump and/or a rate of change of the volume flow rate can also be taken into account in the differential element.

The differential element is advantageously set to 0 in certain pressure phases. In this way, a prospective control intervention can be switched off if necessary.

In addition, the control device may comprise a control system for smoothing pressure peaks of the pressure curve in pressure cycles. The control system is advantageously a speed adapter.

The speed adapter is preferably set up in such a way that, in each case at a specific progress of the pressure cycle, a set speed of a delivery element of the peristaltic pump is lowered by a specific amount to a lowered set speed compared to an average set speed before completion of a pressure cycle and/or is increased by a specific amount to a higher setpoint speed being higher that the average setpoint speed after completion of a pressure cycle and is held for a specific holding time.

Since the pressure—and thus the necessary delivery power—drops rapidly during a transition from one pressure cycle to the next, the power requirement of the peristaltic pump also changes abruptly. In order to maintain a constant target volume flow rate, the power of the peristaltic pump would therefore have to be reduced or even reversed (braked) during the transition between a preceding and the following pressure cycle. This is not only disadvantageous in terms of energy. With the proposed control system, a brief increase in the rotational speed of the peristaltic pump, or the delivery elements respectively, a briefly increased maximum volume flow rate is permissible when changing the pressure cycle. By increasing the target volume flow rate or the rotational speed at the beginning of a pressure cycle, the pressure drop can be reduced and a pressure level required for the injection can be reached more quickly afterwards. By reducing the target volume flow rate and the rotational speed at the end of a pressure cycle, the pressure increase can be stretched over time. This creates additional safety reserves.

The speed of the conveying elements increases abruptly to the increased set speed after completion of a pressure cycle and is then reduced linearly to the reduced set speed of the conveying element after the holding time. In this way, peak loads can be reduced.

All holding times can be the same or different. The increase and decrease of the set speed is expediently equal in amount. The speed of the peristaltic pump can, for example, be increased or decreased in percentage terms by a predetermined proportion of the mean setpoint speed, whereby the change in speed is preferably between 20% and 40% of the mean setpoint speed and in particular can be 20%, 30% or 40% above or below the mean setpoint speed.

It is particularly preferred if the control system is superimposed on the first control loop via a speed adapter. In this way, the change caused by the control system can be taken into account as a modified command variable of the setpoint rotational speed in the first control loop. In the context of these considerations, the rotational speed and the setpoint volumetric flow rate are regarded as directly proportional to each other and therefore interchangeable. As has been shown, the influences of the first control loop with the pressure prediction as well as the influences of the control system mutually reinforce each other, so that the control quality of the control device can be disproportionately increased.

The conveying action of a peristaltic pump regularly comprises more than one, preferably more than five and in particular more than ten pressure cycles.

In another aspect of the invention, an injection device for injecting an injection agent into an animal or human body by means of a peristaltic pump in the form of a roller pump is proposed, which has a control device according to the invention for controlling the roller pump. Using injection devices with such a control device, uniform pressure cycles with low pressure amplitudes can be achieved for roller pumps.

In yet another aspect of the invention, a control method is proposed for a peristaltic pump comprising a squeeze tube and cyclically moving conveying elements for conveying a medium guided in the squeeze tube during a conveying action with a controlled volumetric flow rate into a discharge line connected to the squeeze tube, wherein the conveying elements cyclically compress the squeeze tube so that a pressure curve of a pressure is established in the discharge line, which pressure curve has cyclically repeating pressure cycles, wherein each pressure cycle has a pressure minimum, a pressure rise, a pressure maximum, and a pressure drop, wherein the control method controls a speed of the peristaltic pump in such a way that a maximum volume flow rate is achieved without exceeding a pressure limit in the discharge line, wherein the control method comprises a first control loop for controlling the maximum volume flow rate, which receives a target volume flow rate and the pressure limit as command variables, and for each pressure cycle a predicted pressure is calculated for an expected maximum pressure within the pressure cycle on the basis of the pressure in the discharge line. The maximum volume flow rate is then limited, taking into account the predicted pressure, so that the pressure in the output line does not exceed the pressure limit in the pressure cycle.

Further features and advantageous embodiments of the invention are apparent from the subclaims and the following description, in which the invention is described by way of exemplary embodiments with reference to the accompanying figures, reference being made in each case equally to the control device according to the invention, the method according to the invention and the roller pump and injection device according to the invention.

Thereby show

FIG. 1: a schematic representation of a contrast medium injection device with a roller pump,

FIG. 2: A detailed illustration of a roller pump with conveying elements,

FIG. 3: A diagram of a typical pressure curve of a roller pump over time, as well as the associated pressure phases and a curve of the angle of rotation of the conveying elements over time,

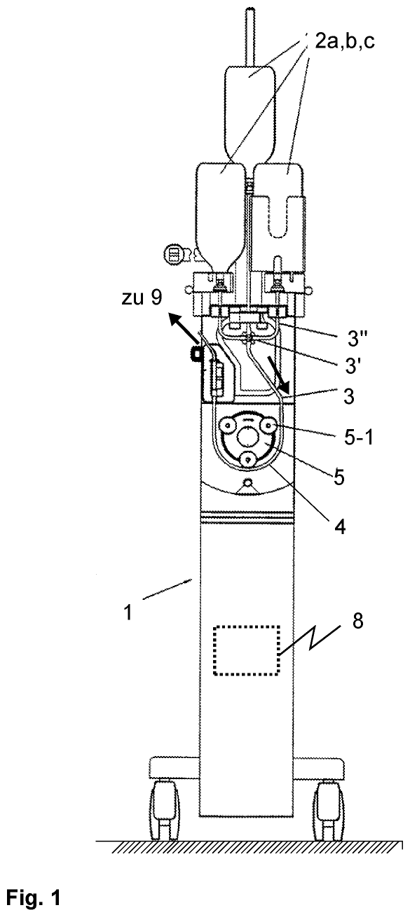

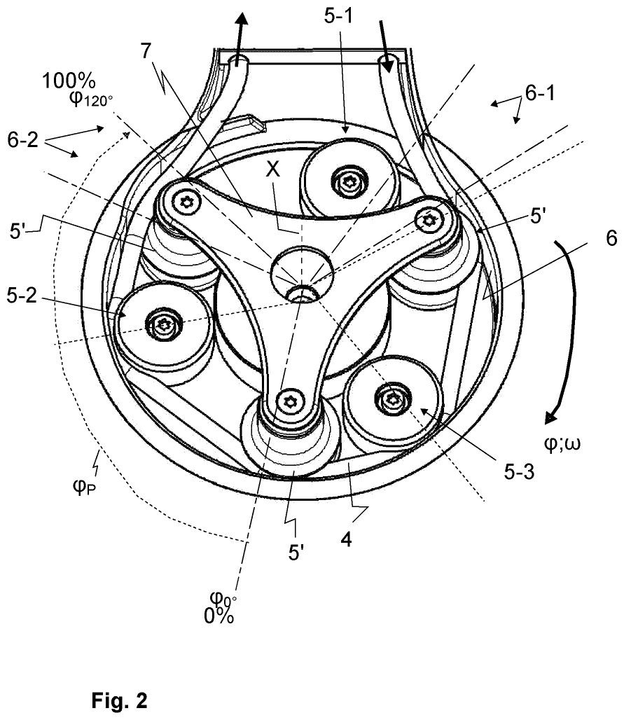

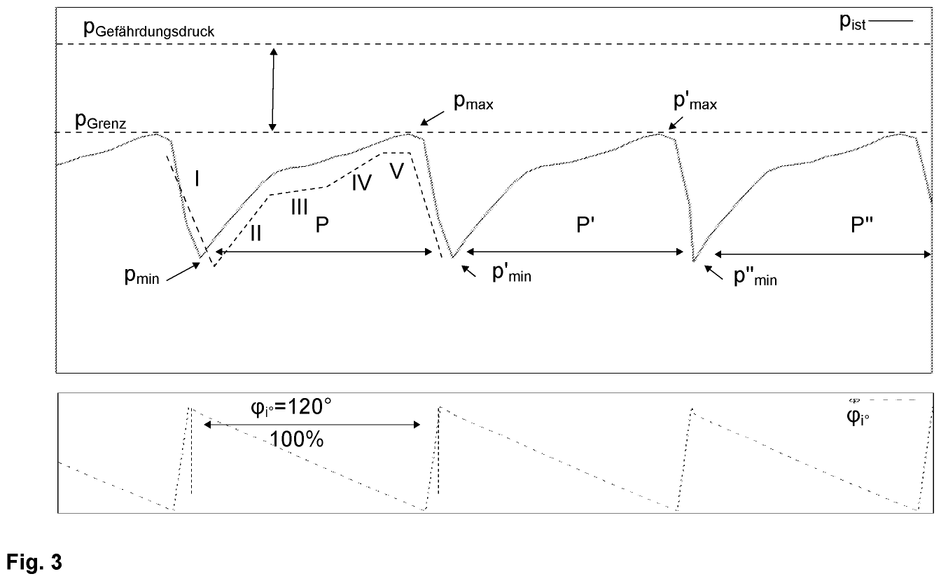

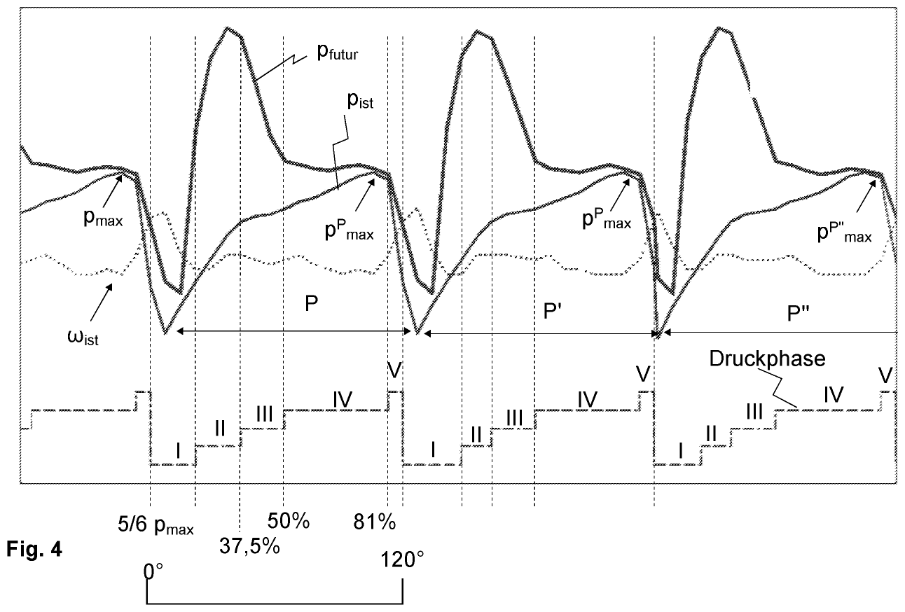

FIG. 4: A diagram of a typical pressure curve of a roller pump controlled according to the invention with phase detection as well as pressure prediction,

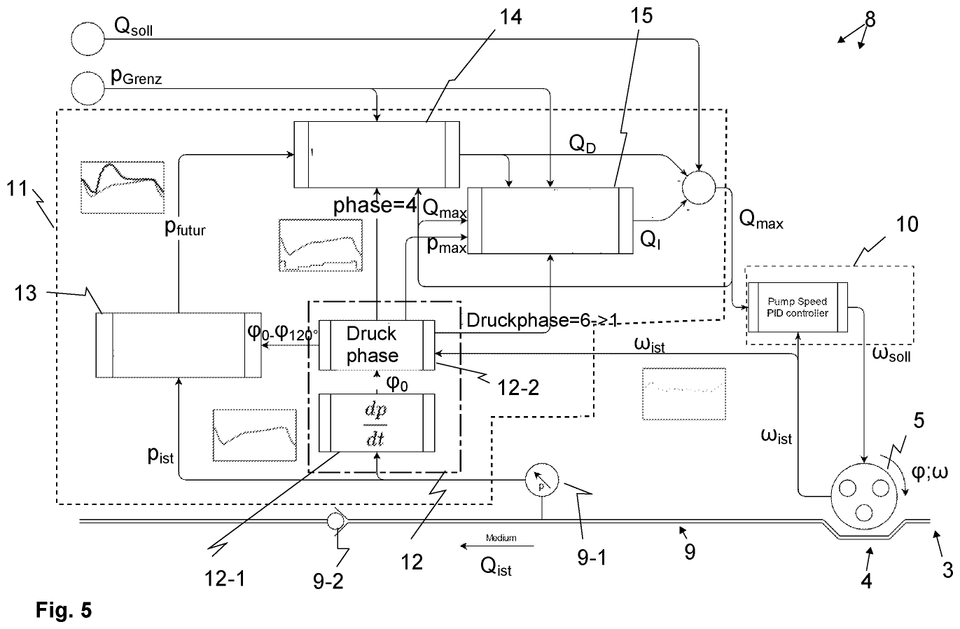

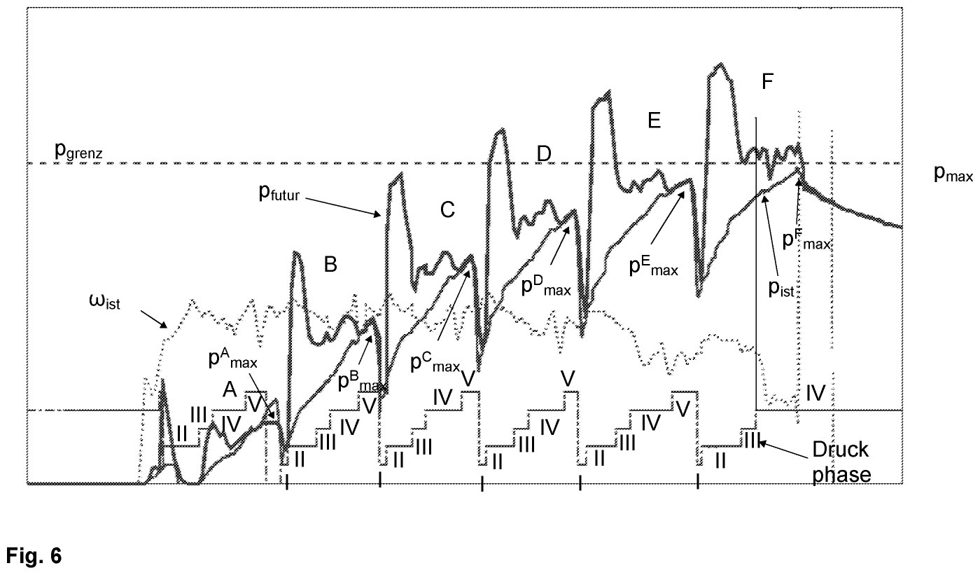

FIG. 5: A circuit diagram of a control device according to the invention,

FIG. 6: A pressure curve with occluded discharge line with control response of the control device from FIG. 5,

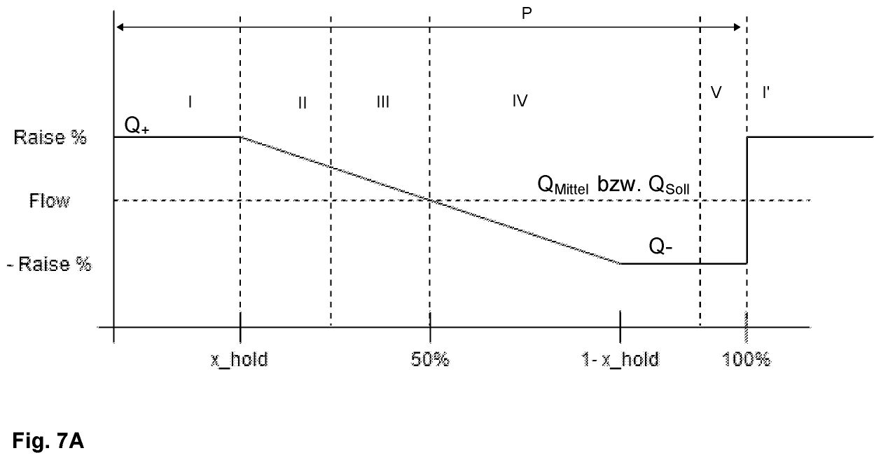

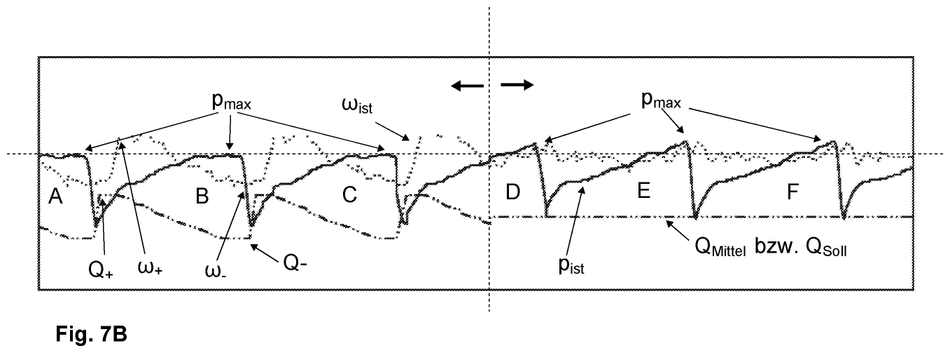

FIG. 7A: a diagram for a pressure phase-dependent control of a maximum volume flow rate in a control device according to the invention, as well as

FIG. 7B: A diagram of pressure curve, volume flow rate and angular velocity for a roller pump with a volume flow rate control according to FIG. 7A (left) and without volume flow rate control (right).

In the figures, identical or comparable components, functions or elements are given the same or comparable reference signs. If reference signs are used repeatedly, referral is made to the respective previous description.

FIG. 1 shows an injection device 1 for the intravenous administration of an injection agent. The injection device 1 has a base body mounted on rollers, at the upper end of which several injection agent containers 2a, 2b, 2c are arranged. Contrast media and a saline solution are stored in the injection medium containers 2a, 2b, 2c. The injection medium containers 2a, 2b, 2c are connected to a squeeze tube 4 via a supply line 3. The squeeze tube 4 is inserted into the tube bed 6 of a roller pump 5 and is connected on the outlet side to a discharge line 9, which in turn can be connected to the bloodstream of a patient via a catheter by means of an adjoining patient tube (not shown).

The roller pump 5 has three rollers 5-1, 5-2, 5-3 rotatably mounted on an electric motor-driven rotor 7, which are guided along the tube bed 6 of the peristaltic pump 5 as shown in FIG. 2, and in doing so squeeze the squeeze tube 4 in certain areas against a counter-bearing part of the tube bed 6, so that when the rotor 7 rotates about its axis of rotation, an injection medium in the squeeze tube 4 is conveyed in portions into the outlet line 9 against a back pressure in the outlet line 9. The injection device 1 has a control device 8 for controlling the roller pump 5 (and further components of the injection device 1).

FIG. 2 shows a detailed perspective view of the roller pump 5. As can be seen in FIG. 2, the squeeze tube 4 runs over an angular range of approximately 260°—with respect to the axis of rotation X of the roller pump 5—between an inlet area 6-1 of the tube bed 6 and an outlet area 6-2 of the tube bed 6. Since the three rollers 5-1, 5-2, 5-3 are arranged evenly at an angular distance of 120° each on the rotor 7, there are thus always at least two rollers between the inlet area 6-1 and the outlet area 6-2 of the tube bed 6, which compress the squeeze tube 4 against the counter bearing part of the tube bed 6. The squeeze tube 4 is therefore always compressed in at least two places during operation of the roller pump.

The rotor 7 of the peristaltic pump rotates in a direction of rotation—clockwise in the illustration of FIG. 2—so that the compressed areas of the squeeze tube 4 with the rollers 5-1, 5-2, 5-3 move in the direction of rotation. Between two compressed areas, a fluid-tight tube section with a certain volume is formed in each case, so that injection medium contained therein is encapsulated between two rollers 5-1, 5-2, 5-3 and moved in the direction of rotation to the outlet area 6-2. When in the outlet area 6-2 a roller 5-1 starts to lift off from the tube bed 6, the injection medium can be conveyed in the tube section against the back pressure prevailing in the remaining part of the discharge line 9. In FIG. 2, the roller 5-1 has already completely left the outlet area 6-2 in the direction of rotation (the squeeze tube 4 is therefore no longer compressed by the roller 5-1), and the injection agent is conveyed in the tube section against the dynamic pressure prevailing in the remaining part of the discharge line 9.

A pressure sensor 9-1 shown in FIG. 5 is arranged in the discharge line 9, which continuously measures the pressure pᵢₛₜ prevailing therein and transmits it to the control device 8.

The cyclically exiting of a roller 5-1 in the outlet area 6-2 results in a characteristic pressure curve of the pressure pᵢₛₜ with cyclically repeating pressure cycles P in the discharge line 9. A characteristic pressure curve pᵢₛₜ with several pressure cycles P, P′, P″ is exemplarily shown in FIG. 3 at the top in its temporal course. During a pressure cycle P, the pressure fluctuates between a pressure minimum pₘᵢₙ at the beginning of the pressure cycle P, rises rapidly at first, then passes into a range with a moderate pressure increase and reaches a brief pressure plateau or pressure maximum pₘₐₓ before the pressure drops rapidly and the next pressure cycle P′ begins.

The beginning of a pressure cycle P correlates with the exit of the first roller 5-1 from the tube bed 6 in the outlet area 6-2, at which time a second roller 5-2 is in the angular position marked in FIG. 2 with the reference sign φ_{0°} This second roller 5-2 represents the leading roller at this moment (and until it exits via the exit area 6-2) and, with further rotation, continues to reduce the tube volume of the squeeze tube 4 in front of it against a back pressure in the discharge line 9, which leads to the characteristic pressure increase. When this second roller leaves the tube bed 6 in the outlet area 6-2, i.e. approximately at an angular position which is provided with the reference sign φ_{120°} in FIG. 2, the compression of the squeeze tube 4 at this region is terminated, so that the volume increases approximately abruptly up to the subsequent third roller 5-3. This leads to the characteristic pressure drop at the end of a pressure cycle P.

As can be seen in FIG. 3, the pressure curve at the beginning of the pressure cycle is comparatively steep (range II) as long as a non-return valve 9-2 (see FIG. 5) in the discharge line 9 is still closed. As soon as the closing pressure of the non-return valve 9-2 is reached, the non-return valve 9-2 opens and the pressure increase drops (range III). At this point, the liquid column of the injection medium present in the discharge line 9 and the patient tube following downstream must be set in motion against the mass inertias as well as further throttle resistances, which is responsible for the further pressure increase. A further pressure increase occurs in range IV, which is due to another non-return valve in a catheter. After the pressure resistances have been overcome, a quasi-static flow state with an approximately constant pressure (range V) is established before the pressure drops again when a roller exits the tube bed 6 (range I).

During a complete revolution of the rotor 7, each of the three rollers 5-1, 5-2, 5-3 will therefore pass once through the angular range designated φ_{P} between φ_{0°} and φ_{120°} and cause a pressure cycle P. The angular range φ_{P} thus comprises angles φ_{i°} between 0° and 120°, designated here as the pressure phase angle, where the subscript i stands for the angle. It is evident that the pressure phase angle φ_{i°} is directly related to the angular position φ of the rotor 7 or the individual rollers via the modulo relationship φ_{i°}=φ mod 120°.

For clarification, a time course of the pressure phase angle φ_{i°} is drawn in FIG. 3 below.

In order to guide an actual volume flow rate Qᵢₛₜ in the discharge line 9 as close as possible to a specified target volumetric flow, it is necessary to guide the actual pressure curve of the pressure pᵢₛₜ as close as possible to the pressure limit p_{Grenz} that is still permissible. In the present embodiment example of the invention, the roller pump 5 is to be controlled to a maximum volume flow rate Qₘₐₓ without exceeding the still permissible pressure limit p_{Grenz}. The pressure limit p_{Grenz} can therefore also be understood as the (maximum) set pressure for the pressure pᵢₛₜ. Above the limit value p_{Grenz}. FIG. 3 also shows a hazard pressure p_{Gefährdungsdruck}, at which irreparable damage to the device or patient occurs. It must therefore be ensured that the hazard pressure p_{Gefährdungsdruck} is not exceeded under any circumstances.

Unlike syringe pumps, where a piston is pushed into a cylinder at a controlled speed, it is much more difficult to maintain the pressure limit p_{Grenz} with roller pumps because of the pressure pulsations. For this reason, high safety buffers (distance between p_{Grenz} and p_{Gefährdungsdruck}) must be usually incorporated in known roller pumps in order to prevent the hazard pressure p_{Gefährdungsdruck} from being reached or even exceeded in the further course of a pressure cycle if the pressure limit p_{Grenz} is already exceeded at the beginning of a pressure cycle.

To compensate for this system-related disadvantage, the control device 8 of this embodiment provides a control according to the control scheme shown in FIG. 5. The control device 8 comprises a second control loop 10 for controlling the rotational (angular) speed ω of the rotor 7 of the roller pump 5, the pump speed PID controller, which receives the maximum volume flow rate Qₘₐₓ as a command variable and converts this into a set rotational (angular) speed ω_{Soll} or a corresponding rotating field for controlling the roller pump. The actual rotational (angular) speed ωᵢₛₜ is fed back as a measured variable and a control deviation between the set and actual rotational (angular) speed is minimised by feedback.

To determine the maximum volume flow rate Qₘₐₓ, a first control loop 11 is connected upstream of the second control loop 10, to which a setpoint volumetric flow rate Q_{Soll} and a permissible pressure limit p_{Grenz} are externally specified as command variables. The setpoint volumetric flow rate Q_{Soll} (unchanged), the pressure pᵢₛₜ measured in the discharge line 9 and the angular speed ωᵢₛₜ of the rotor 7 of the roller pump 5 measured at the roller pump 5 enter the first control loop 11 as feedback variables. The measurement and feedback of an actual volume flow rate Qᵢₛₜ in the discharge line 9 can be dispensed with.

The first control loop 11 is designed as a discontinuous PID controller that regulates the controlled variable of the maximum volume flow rate Qₘₐₓ differently depending on the pressure phase of a pressure cycle.

The first control loop 11 comprises a pressure phase detection 12 including a pressure phase commutation detector 12-1 and a pressure phase switch 12-2, a pressure prediction 13, and a first control component 14 and a second control component 15.

The pressure phase commutation detector 12-1 is used to detect the start of a pressure phase. For this purpose, it continuously compares the current pressure pᵢₛₜ with a reference value, namely a temporarily stored pressure maximum pₘₐₓ of a previous pressure cycle P, and sets the start of a pressure cycle P to the point in time at which a pressure drop of the pressure pᵢₛₜ in the discharge line 9 of more than ⅙, when compared to the temporarily stored pressure maximum pₘₐₓ, occurs. At this time, one of the rollers 5-1, 5-2, 5-3 of the roller pump 5 is approximately in the angular position marked φ_{0°} in FIG. 2. The pressure phase angle φ_{i°} is set to zero, i.e. φ_{0°}=0°, and serves in the further course of the pressure cycle P as a reference value for determining subsequent pressure phases. “Phase I” is set as the current pressure phase.

The pressure phase switch 12-2 switches or counts up the individual pressure phases II to V. The individual pressure phases or the start of the individual pressure phases are determined via the angle φ_{i°}. The pressure phase angle φ_{i°} in turn is detected or approximated—starting from the reference value φ_{0°}—via a time integration of the actual angular velocity of rotation of the roller pump.

The different pressure phases I to V are defined as follows:

The beginning of the first pressure phase I is defined by a pressure drop of more than ⅙ compared to the pressure maximum of the previous pressure cycle. The beginning of the second pressure phase II is set to the point in time at which or after a pressure increase is certain. The third pressure phase III corresponds to a first compression phase, the beginning of which is defined at a pressure phase angle of 45° (this corresponds to a phase progress of 37.5% of the complete angular range φ_{P}). A fourth pressure phase IV is defined at a pressure phase angle of 60° (phase progress of 50%). The beginning of the fifth pressure phase V is set at a pressure phase angle of 97.2° (phase progress of 81%).

The individual pressure phases I to V of several successive pressure cycles P, P′, P″ can be taken from FIG. 4.

The first control component 14 is activated via the pressure phase detection 12 when the current pressure cycle P is in the fourth pressure phase IV. In all other pressure phases (I to III and V), the first control component 14 is deactivated, i.e. set to zero.

The first control component 14 is the differential element of the PID controller. In it, a correction factor Q_{D} is calculated, which is deducted from the target volume flow rate Q_{Soll}. It is calculated as the ratio of a predicted pressure p_{futur} (or p_{forecast}) and the pressure limit p_{Grenz} multiplied by the currently calculated maximum volume flow rate Qₘₐₓ according to the formula

$${Q}_{D} = {K}_{D} \frac{{p}_{futur}}{{p}_{Grenz}} {Q}_{max} ,$$

where K_{D} is a fixed gain factor.

The predicted pressure p_{futur} is continuously provided by the pressure prediction 13. The predicted pressure p_{futur} is determined as a linear extrapolation of the current pressure pᵢₛₜ to a defined pressure phase angle of 105° (or a phase progress of 87.5%) according to the formula

$${p}_{futur} = {p}_{ist} \frac{{dp}_{ist}}{dt} \frac{φ t}{φ87 , 5 \%} ,$$

$$where$$

$$\frac{{dp}_{ist}}{dt}$$

is the time derivative of the measured pressure pᵢₛₜ and

$$\frac{φ}{φ87 , 5 \%}$$

determines the ratio of the current pressure phase angle φᵢ₌ₜ at time t and the pressure phase angle at a phase progress of 87.5% (this corresponds to a pressure phase angle of 105° for a complete angular range φ_{P} of 120°). The predicted pressure represents a forecast for the expected pressure maximum in the current pressure cycle at the defined pressure phase angle. The predicted pressure p_{futur} is plotted in its temporal course in FIG. 4.

The terms “prediction pressure” and “predicted pressure” are to be understood synonymously.

The second control component 15 is the proportional and integral element of the PID controller. In it, a correction factor Q_{I} is calculated, which is deducted from the setpoint volume flow rate Q_{Soll} and is calculated via three components α, β, γ:

The first component α of the second control component 15 is a mean value filter which adjusts the maximum volume flow rate Qₘₐₓ via the ratio of the maximum pressure pₘₐₓ of the immediately preceding pressure cycle P compared to the pressure limit p_{Grenz}, multiplied by a gain factor K_{I}, according to the formula

$$α = {K}_{I} \frac{{p}_{max}}{{p}_{Grenz}} {Q}_{max} .$$

The maximum pressure pₘₐₓ of each pressure cycle is temporarily stored in a memory in the control unit 8 for this purpose. Measurement errors of the pressure pᵢₛₜ can be smoothed via the mean value filter.

The second component β of the second control component 15 is a proportional element which increases the correction factor Q_{D} calculated in the previous pressure cycle P by the first control component 14 by a gain factor K_{D→I} according to the formula

β=K_{D→I}Q_{D}.

Since the first control component 14 calculates the correction factor Q_{D} only in pressure phase IV, the second component β of the second control component 15 is zero when the pressure cycle is outside pressure phase IV.

The third component γ of the second control component 15 is an integral element that is calculated from the sum of the previous volume flow rate corrections Q_{I n-1} of the individual pressure phases of the current pressure cycle, according to the formula

γ=Σₙ₋₁Q_{I n-1},

where n is the number of pressure cycles and Q_{I n-1} is the volume flow rate correction of successive pressure cycles.

The three components α, β, γ of the second control component are added up to a correction variable Q_{I} (Q_{I}=α+β+γ) and the correction variable Q_{I} is subtracted from the setpoint volume flow rate Q_{Soll}.

Thus, the maximum setpoint volume flow rate Qₘₐₓ results from the setpoint volume flow rate Qₛₒₗₗ minus the correction variables Q_{D} and Q_{I} of the first and second control components 14 and 15 (Qₘₐₓ=Q_{Soll}−Q_{I}−Q_{D}).

Via the control device 8 designed in this way, the output of the roller pump 5 or its rotational speed ω can be regulated and adjusted in a prospective manner. Since the maximum volume flow rate Qₘₐₓ is calculated differently in pressure phases I to III and V than in pressure phase IV, this is a discontinuous control.

A control response for the case of a pinched-off, fluid-impermeable discharge line 9 is shown in the diagram of FIG. 6 with six successive pressure cycles A to F showing the measured pressure pᵢₛₜ, the predicted pressure p_{futur} calculated by the pressure prediction 13 and the associated pressure phases I to V of the individual pressure cycles A to F. As a result of the disconnected discharge line 9, the absolute pressure maximum pₘₐₓ increases steadily in each pressure cycle: p^{A}ₘₐₓ<p^{B}ₘₐₓ<p^{C}ₘₐₓ<p^{D}ₘₐₓ etc.

The predicted pressure p_{futur} exceeds the pressure limit p_{Grenz} for the first time in a fourth pressure phase IV in pressure cycle F. The other exceedances of the predicted pressure p_{futur} beyond the pressure limit p_{Grenz} in other phases, e.g. in pressure cycle D in pressure phase II, are irrelevant, since the control device 8 only takes the predicted pressure p_{futur} into account in pressure phase IV via the control component 14.

Since every fourth pressure phase IV already begins at a pressure phase angle of 60° (or a phase progress of 50%), the maximum setpoint volume flow rate Qₘₐₓ in the pressure cycle F and thus also the rotational speed ωᵢₛₜ are reduced by the control device 8 with the onset of the pressure phase IV in the pressure cycle F. The throttling of the rotational speed ω at the beginning of phase IV is clearly visible in FIG. 6. The pressure limit p_{Grenz} is not exceeded due to the sufficient lead time between the detection of an imminent exceeding of the pressure limit p_{Grenz} and the occurrence of the pressure maximum p^{F}ₘₐₓ<p_{Grenz}.

In order to achieve a levelling of the pressure curve of the pressure pᵢₛₜ, i.e. a reduction of the amplitude, the control device 8 of the embodiment example according to FIG. 5 can also be supplemented by a control system not shown in FIG. 5, which increases or decreases the maximum setpoint volume flow rate Q_{Soll} depending on the phase progress. This control manipulates the command variable of the setpoint volumetric flow rate Q_{Soll} in such a way that the setpoint volumetric flow rate Q_{Soll} is increased by 30% to an increased setpoint volumetric flow rate Q₊ at the beginning of a pressure cycle and is reduced by 30% to a reduced setpoint volumetric flow Q₋ towards the end of a pressure cycle. The changed setpoint flow rate Q_{Soll} is held for a holding time of x-hold. The holding time x-hold can correspond to a certain phase progress, such as e.g. 25% or the duration of phase I. Due to the opposite adjustment to the increased or decreased setpoint volumetric flow rate Q₊ or Q₋, the average setpoint volumetric flow rate over a pressure cycle P remains unchanged. The corresponding presetting of the target volumetric flow rate is shown in FIG. 7A for a pressure cycle P with pressure phases I to V. As can be seen in FIG. 7A, the set volumetric flow rate Q_{Soll} is suddenly increased to the set volumetric flow Q₊ at the beginning of the pressure cycle P and then decreases linearly to the reduced set volumetric flow rate Q₋. The command variable of the setpoint volumetric flow rate Q_{Soll} changed in this way, which is now variable in time over a pressure cycle P, is fed into the first control loop 11.

Such control results in a smoother pressure curve with fewer pressure fluctuations and, in particular, flattened pressure peaks. This is shown in FIG. 7B: FIG. 7B shows a pressure curve for several pressure cycles A to F, whereby the setpoint volume flow rate Q_{Soll} in the pressure cycles A to C and the beginning of the pressure cycle D was adjusted according to the variable setpoint volume flow rate Q_{Soll} according to FIG. 7A, and a constant setpoint volume rate flow was specified in the subsequent pressure cycles. As can be seen in FIG. 7B, the pressure peaks pₘₐₓ of the pressure cycles A, B and C can thus be reduced compared to the pressure peaks pₘₐₓ of the pressure cycles D, E and F.

Another effect of this control is also that a motor power of the peristaltic pump 5 after the sudden pressure drop at the end of a pressure cycle P does not have to be abruptly reduced or even slowed down to keep the volume flow rate constant. This can save energy and reduce motor noise.

Since the setpoint volumetric flow rate Q_{Soll} is approximately directly proportional to the angular velocity ωᵢₛₜ of the peristaltic pump 5, the angular velocity ωᵢₛₜ of the peristaltic pump 5 (discounting for measurement inaccuracies and the influence of the controlled system) has a comparable curve to the target volume flow rate Q_{Soll}. It is therefore possible to increase the angular velocity ω_{Soll} of the peristaltic pump to an increased setpoint velocity ω₊ at the beginning of a pressure cycle and/or to decrease it to a lower setpoint velocity ω₋ towards the end of the pressure cycle instead of the setpoint volumetric flow rate.

The invention exemplified in the described embodiments enables safer operation of a peristaltic pump with higher media throughput and lower pressure fluctuations.

In addition to peristaltic pumps, the control device can also be used for other pumps with cyclically repeating pressure curves, such as diaphragm pumps with an oscillating diaphragm, piston pumps with reciprocating pistons, sine pumps or gear pumps. The control device is particularly suitable for application purposes in which overpressure valves, over which medium is set free, are incompatible with the system to be used in.

### LIST OF REFERENCE SIGNS

I to V Pressure phases of a pressure cycle
p_{Grenz} Pressure limit (target pressure)
p_{futur} Prediction pressure
p_{Gefährdungsdruck} Hazard pressure
P Pressure cycle (period)
Qₘₐₓ maximum volume flow rate
Qₛₒₗₗ Target volume flow rate/setpoint volumetric flow rate
X Rotary axis (roller pump)
φ_{i°} Pressure phase angle (at an angle i)
ωᵢₛₜ Rotation angle speed of the roller pump/rollers 5-1.5-2, 5-3 (actual value)
ωₛₒₗₗ Angular velocity of rotation (set point)
1 Injection device
2a-c Injection medium container
3, 3′, 3″ Supply line
4 Squeeze tube
5 Peristaltic pump (roller pump)
5-1 Roll (squeeze roll)
5-2 Roll (squeeze roll)
5-3 Roll (squeeze roll)
5′ leading roll
6 Tube bed
6-1 Inlet area
6-2 Outlet area
7 Rotor
8 Control device
9 Discharge line
9-1 Pressure sensor (injection line)
9-2 Non-Return valve (injection line)
10 Second control loop (pump speed controller)
11 First control loop
12 Pressure phase detection
12-1 Pressure phase commutator
12-2 Pressure phase switch
13 Pressure predictor
14 first control component
15 second control component

1. A control device for a peristaltic pump with a squeeze tube and cyclically moving conveying elements for conveying a medium guided in the squeeze tube into a discharge line connected to the squeeze tube during a conveying action with a controlled volume flow rate,
wherein the conveying elements are adapted to cyclically compress the squeeze tube so that a pressure curve of a pressure (pᵢₛₜ) is established in the discharge line, the pressure curve having cyclically repeating pressure cycles (P),
wherein each pressure cycle (P) has a pressure minimum, a pressure rise, a pressure maximum and a pressure drop,
wherein the control device is adapted to controls a speed of the peristaltic pump in such a way that a maximum volume flow rate (Qₘₐₓ) is achieved without exceeding a pressure limit (p_{Grenz}) in the discharge line,
and wherein the control device has a first control loop for controlling the maximum volume flow rate (Qₘₐₓ), adapted to receives a setpoint volumetric flow (Qₛₒₗₗ) and the pressure limit (p_{Grenz}) as command variables, and is arranged such that
for each pressure cycle (P), a prediction pressure (p_{futur}) is calculated for an expected maximum pressure within the pressure cycle (P) on the basis of at least the pressure (pᵢₛₜ) in the discharge line,
and the maximum volume flow rate (Qₘₐₓ) is limited, taking into account the prediction pressure (p_{futur}), in such a way that the pressure (pᵢₛₜ) in the discharge line does not exceed the pressure limit (p_{Grenz}) in the pressure cycle (P).

2. The control device according to claim 1, wherein the first control loop comprises a pressure phase detection which detects the end of a preceding pressure cycle (P) and the beginning of a subsequent pressure cycle (P′) on the basis of a characteristic variable, in particular a defined pressure drop relative to a pressure maximum (pₘₐₓ) of the preceding pressure cycle (P), in order to initiate the control of the maximum volume flow rate (Qₘₐₓ) for the subsequent pressure cycle (P).

3. The control device according to claim 1, wherein each pressure cycle (P) is divided into successive pressure phases (I to V) on the basis of predefined characteristics, in particular a pressure change and/or a rate of change of the pressure (pᵢₛₜ),
and the pressure phase detection detects the start of the individual pressure phases (I to V) either on the basis of the predefined characteristics and/or on the basis of a position of the conveying elements of the peristaltic pump,
and the predicted pressure (p_{futur}) for regulating the maximum volume flow rate (Qₘₐₓ) in the first control loop is used only in defined pressure phases and is ignored in other pressure phases.

4. The control device according to claim 3, wherein a first pressure phase (I) is characterised by a rapid pressure loss, a second pressure phase (II) by a rapid pressure rise, a third pressure phase (III) by a mild pressure rise, a fourth pressure phase (IV) by a moderate pressure rise (IV) and a fifth pressure phase (V) by a pressure plateau with substantially constant pressure, and each pressure phase is detected by the pressure phase detection.

5. The control device according to claim 4, wherein in the first control loop, the prediction pressure (p_{futur}) is used to limit the maximum volumetric flow rate (Qₘₐₓ) only in the fourth pressure phase (IV), wherein the fourth pressure phase (IV) preferably and approximately is extending in a range of the pressure cycle (P) of 50% to 80% of a phase progress of the pressure cycle (P).

6. The control device according to claim 2, wherein the pressure phase detection determines the start of at least one pressure phase of a pressure cycle (P) on the basis of a pressure change (Δp) of the pressure (pᵢₛₜ) and/or a pressure change rate (dp/dt) of the pressure (pᵢₛₜ).

7. The control device according to claim 2, wherein in the control method, the predicted pressure (p_{futur}) is determined as a linear extrapolation of the current pressure increase (pᵢₛₜ) to a specific, calculated or predetermined phase progress (φ_{87%}) of the pressure cycle (P).

8. The control device according to claim 7, wherein in the control method, the predetermined or calculated phase progress (φ_{87%}) at which the prediction pressure (p_{futur}) is determined does not coincide with an actual phase progress (φ_{81%}) at which the actual maximum pressure (pₘₐₓ) of a pressure cycle (P) occurs.

9. The control device according to claim 1, wherein the first control circuit comprises a mean value filter which adjusts the maximum volume flow rate (Qₘₐₓ) as a function of a maximum pressure (pₘₐₓ) of a pressure cycle (P) immediately preceding the current pressure cycle (P) which is temporarily stored in the control device.

10. The control device according to claim 3, wherein the first control loop is a PID controller with a proportional element (P), an integral element (I) and a differential element (D), preferably the differential element being set to zero in certain pressure phases, in particular in the first pressure phase (I), the second pressure phase (II), the third pressure phase (III) and the fifth pressure phase (V).

11. The control device according to claim 1, wherein the control device comprises a control system for smoothing pressure peaks of the pressure curve of the pressure (pᵢₛₜ) in the individual pressure cycles (P),
said control system being arranged as a speed adapter, such that in each case at a specific progress of the pressure cycle, a setpoint speed (ω_{Soll}) of a conveying element of the peristaltic pump is lowered by a specific amount to a lower setpoint speed (ω−) with respect to an average setpoint speed before completion of a pressure cycle (P) and/or is increased by a specific amount to a higher setpoint speed (ω₊) being higher than the average setpoint speed after completion of a pressure cycle (P), and is held for a specific holding period.

12. The control device according to claim 11,
wherein the speed (ω) of a conveying element is increased abruptly by the defined amount after completion of a pressure cycle (P) and is then reduced linearly to the lower setpoint speed (ω−) of the conveying element.

13. The control device according to claim 1, wherein the conveying process comprises more than one, preferably more than 5 and particularly more than 10 pressure cycles.

14. A peristaltic pump in the form of a roller pump with a rotatable rotor and a control device according to claim 1, wherein
the conveying elements are designed as rollers arranged on a rotor,
the squeeze tube is supported along a tube bed with an inlet region and an outlet region,
wherein the conveying elements are configures to, upon rotation of the rotor, successively compress in a fluid-tight manner a section of the squeeze tube as the conveying elements enter the hose bed at the level of the inlet region until the conveying elements exit the tube bed at the level of the outlet region and
thereby the medium located in the squeeze tube is conveyed into the discharge line against a dynamic pressure arising in the discharge line,
wherein each time a volume of the squeeze tube located upstream of the outlet region is compressed from a maximum volume to a minimum volume until a conveying element emerges from the tube bed at the level of the outlet region and
thereby the compressed section of the squeeze tube compressed by this conveying element is released,
whereby each time the volume located upstream of the outlet region of the squeeze tube is increased from the minimal volume to the maximal volume,
whereby a volume reduction and a subsequent volume increase of the volume located upstream of the outlet region form a complete pressure cycle (P),
wherein the roller pump has n conveying elements, so that the roller pump executes n pump strokes with n pressure cycles (P) during a complete 360° turn of the rotor,
and wherein each pressure phase (I to V) of a pressure cycle (P) corresponds substantially to a certain angular range of an angular position (φ) of a conveying element.

15. The pump according to claim 14, wherein the start of at least one pressure phase (I to V) of a pressure cycle (P) is determined indirectly by the pressure phase detection via an angular position (φ) of a conveying element.

16. An injection device for injecting an injection medium into an animal or human body by means of a peristaltic pump in the form of a roller pump, wherein the injection device contains a roller pump according to claim 14.

17. A control method for controlling a peristaltic pump with a squeeze tube and cyclically moving conveying elements for conveying a medium guided in the squeeze tube during a conveying action with a controlled volume flow rate into a discharge line connected to the squeeze tube,
wherein the conveying elements cyclically compress the squeeze tube so that a pressure curve of a pressure (pᵢₛₜ) is established in the discharge line, and
the pressure curve is having cyclically repeating pressure cycles (P),
wherein each pressure cycle comprises a pressure minimum, a pressure increase, a pressure maximum and a pressure drop,
wherein the control method controls a speed of the peristaltic pump in such a way that a maximum volume flow rate (Qₘₐₓ) is achieved without exceeding a pressure limit (p_{Grenz}) in the discharge line,
the control method is comprising a first control loop for controlling the maximum volumetric flow rate (Qₘₐₓ), which receives as command variables a setpoint volumetric flow rate (Qₛₒₗₗ) and the pressure limit (p_{Grenz}),
wherein
for each pressure cycle (P), a prediction pressure (p_{futur}) is calculated for an expected maximum pressure (pₘₐₓ) within the pressure cycle (P) on the basis of the pressure (pᵢₛₜ) in the discharge line,
and the maximum volume flow rate (Qₘₐₓ) is limited, taking into account the prediction pressure (p_{futur}), in such a way that the pressure (pᵢₛₜ) in the discharge line does not exceed the pressure limit (p_{Grenz}) in the pressure cycle (P).
